(19) United States
(12) Patent Application Publication
(10) Pub. No.: US 2021/0304865 A1
(43) Pub. Date: Sep. 30, 2021

(54) EXERCISE PRESCRIPTION APPARATUS AND METHOD

(71) Applicants: HYUNDAI MOTOR COMPANY, Seoul (KR); KIA MOTORS CORPORATION, Seoul (KR)

(72) Inventors: Sung Un KIM, Yongin-si (KR); Jeong Woo NAHM, Yongin-si (KR)

(21) Appl. No.: 16/984,587

(22) Filed: Aug. 4, 2020

(30) Foreign Application Priority Data
Mar. 31, 2020 (KR) 10-2020-0039287

(51) Int. Cl. G16H 20/30; G16H 50/30; G16H 50/50; G16H 50/70; A61B 5/107; A63B 24/00; A61B 5/00; G06T 11/00

(52) U.S. Cl. CPC G16H 20/30; G16H 50/30; G16H 50/50; G16H 50/70; A61B 5/1077; G06T 2210/44; A61B 5/7278; A61B 5/45; A61B 5/7275; A61B 5/744; G06T 11/00; A63B 24/0075

(57) ABSTRACT

An exercise prescription apparatus includes a body shape measurement device that measures a body shape of a user changing with a progress of an exercise based on a previously prescribed exercise guide and a processor that predicts a future body shape of the user based on body shape history information obtained through the measured body shape of the user and to re-prescribe an exercise guide based on a body shape difference between the predicted future body shape and a target body shape.

### CROSS-REFERENCE TO RELATED APPLICATION

The present application claims the benefit of priority to Korean Patent Application No. 10-2020-0039287, filed on Mar. 31, 2020 in the Korean Intellectual Property Office, the entire disclosure of which is incorporated herein by reference.

### TECHNICAL FIELD

The present disclosure relates to an exercise prescription apparatus and a method thereof.

### BACKGROUND

A vision-based body recognition and exercise system which is a system for measuring an appearance and recognizing a posture using an image of a user may provide a service for managing health of the user. In general, a terminal displaying information related to a user's appearance is called a smart mirror system. The user may measure a body shape of the user and input a target body shape to perform health management.

Conventionally, the target body shape is set based on the body shape first measured by the user, a difference between the user's current body shape and the target body shape is continuously monitored, and feedback is provided to inform the user whether a goal is achieved.

The information disclosed in the Background section above is to aid in the understanding of the background of the present disclosure, and should not be taken as acknowledgement that this information forms any part of prior art.

### SUMMARY

The present disclosure has been made to solve the above-mentioned problems occurring in the prior art while advantages achieved by the prior art are maintained intact.

An aspect of the present disclosure provides an exercise prescription apparatus for predicting a user's body shape change based on a body shape change information and exercise history information of the user and prescribing an exercise guide based on a predicted body shape and a method thereof.

The technical problems to be solved by the present inventive concept are not limited to the aforementioned problems, and any other technical problems not mentioned herein will be clearly understood from the following description by those skilled in the art to which the present disclosure pertains.

According to an aspect of the present disclosure, an exercise prescription apparatus includes a body shape measurement device that measures a body shape of a user changing with a progress of an exercise based on a previously prescribed exercise guide and a processor that predicts a future body shape of the user based on body shape history information obtained through the measured body shape of the user and re-prescribes an exercise guide based on a body shape difference between the predicted future body shape and a target body shape.

The processor determines one or more personal requirements of the user to set the target body shape.

The processor extracts basic skeleton information through a measurement of an initial body shape of the user by the body shape measurement device and reflects the extracted basic skeleton information in the target body shape.

The processor confirms a body shape change trend of the user based on the body shape history information and predicts the future body shape based on the confirmed body shape change trend.

The processor predicts the future body shape based on an exercise type, an exercise intensity, an exercise time, an exercise frequency, and an exercise period of the user.

The processor groups at least one other person having a similar body characteristics to a body characteristics of the user with a predetermined ratio or more, and predicts the future body shape based on the body shape history information of the user and the at least one other person which are grouped.

The body characteristics include a dimensional increase/decrease rate for each body part.

The processor predicts a body appearance type of the user using an increase/decrease maximum limit value of a size of each body part, a convergence value, and a prediction value at a predetermined time.

The processor shapes and outputs at least one of the measured body shape, the future body shape, or the target body shape as an avatar.

According to an aspect of the present disclosure, a method of prescribing an exercise includes measuring a body shape of a user which changes with a progress of an exercise based on a previously prescribed exercise guide, predicting a future body shape of the user based on body shape history information obtained through the measured body shape of the user, and re-prescribing an exercise guide based on a difference in body shape between the predicted future body shape and a target body shape.

The measuring a body shape of a user includes determining one or more personal requirements of the user to set the target body shape.

The measuring a body shape of a user further includes measuring an initial body shape of the user to extract basic skeleton information and reflecting the extracted basic skeleton information in the target body shape.

The predicting a future body shape of the user includes confirming a body shape change trend of the user based on the body shape history information and reflecting the confirmed body shape change trend to predict the future body shape.

The predicting a future body shape of the user includes grouping at least one other person having a similar body characteristics to a body characteristics of the user with a predetermined ratio or more, and predicting the future body shape based on the body history information of the user and the at least one other person which are grouped.

The predicting a future body shape of the user includes predicting a body appearance type of the user using an increase/decrease maximum limit value of a size of each body part, a convergence value, and a prediction value at a predetermined time.

The predicting a future body shape of the user includes predicting the future body shape of the user based on an exercise type, an exercise intensity, an exercise time, an exercise frequency, and an exercise period of the user.

The method of prescribing an exercise further includes shaping and outputting at least one of the measured body shape, the future body shape, or the target body shape of the user as an avatar.

### BRIEF DESCRIPTION OF THE DRAWINGS

The above and other objects, features and advantages of the present disclosure will be more apparent from the following detailed description taken in conjunction with the accompanying drawings:

FIG. 1 is a block diagram illustrating an exercise prescription apparatus according to an embodiment of the present disclosure;

FIGS. 2A to 2C are graphs of relationship between a target body shape and a predicted body shape associated with the present disclosure;

FIG. 3 is a flowchart illustrating a method of predicting a user body shape according to an embodiment of the present disclosure;

FIGS. 4A to 4C are diagrams for explaining future body shape prediction depending on exercise according to an embodiment of the present disclosure;

FIGS. 5 and 6 are diagrams for explaining grouping based on body characteristics according to an embodiment of the present disclosure;

FIG. 7 is a flowchart illustrating a method of prescribing exercise according to an embodiment of the present disclosure;

FIG. 8 is a view for explaining an exercise prescription for each body shape characteristics according to an embodiment of the present disclosure;

FIGS. 9A to 9C are graphs illustrating a correlation between body shape characteristics and an exercise type according to an embodiment of the present disclosure;

FIG. 10 is a view for explaining a method of prescribing exercise according to another embodiment of the present disclosure; and

FIG. 11 is a block diagram illustrating a computing system for executing a method of prescribing exercise according to an embodiment of the present disclosure.

### DETAILED DESCRIPTION

Hereinafter, some embodiments of the present disclosure will be described in detail with reference to the exemplary drawings. In adding the reference numerals to the components of each drawing, it should be noted that the identical or equivalent component is designated by the identical numeral even when they are displayed on other drawings. Further, in describing the embodiment of the present disclosure, a detailed description of well-known features or functions will be ruled out in order not to unnecessarily obscure the gist of the present disclosure.

In describing the components of the embodiment according to the present disclosure, terms such as first, second, “A”, “B”, (a), (b), and the like may be used. These terms are merely intended to distinguish one component from another component, and the terms do not limit the nature, sequence or order of the constituent components. Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art, and are not to be interpreted as having ideal or excessively formal meanings unless clearly defined as having such in the present application.

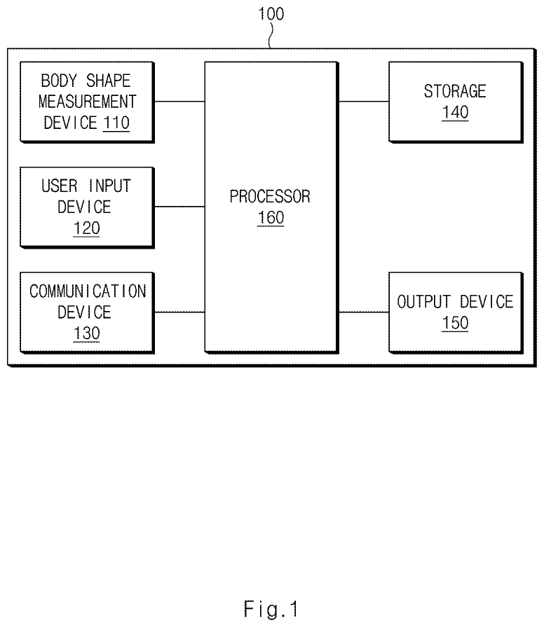

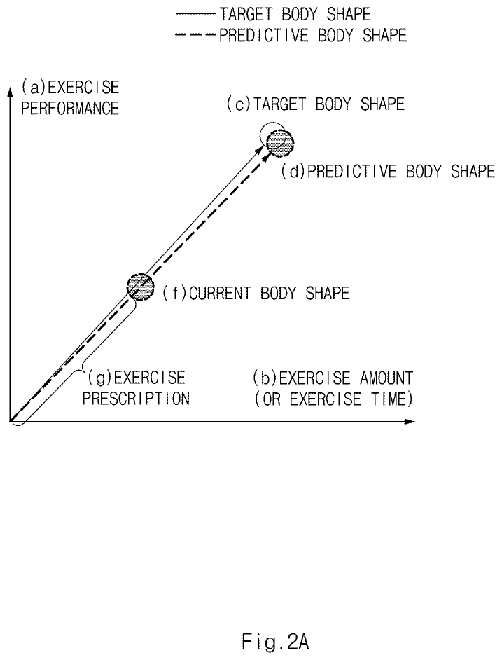

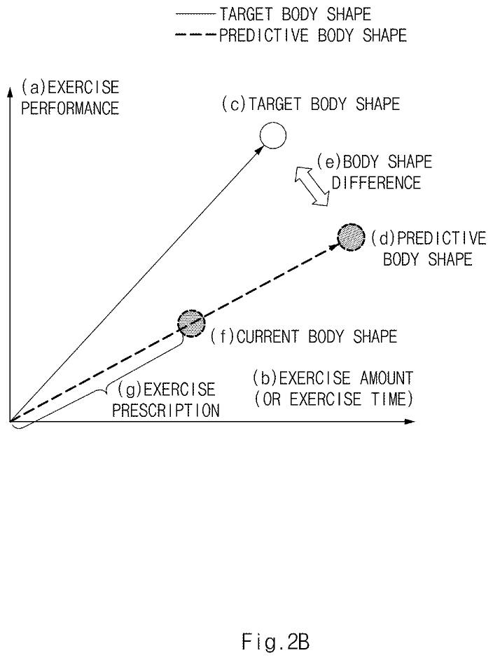

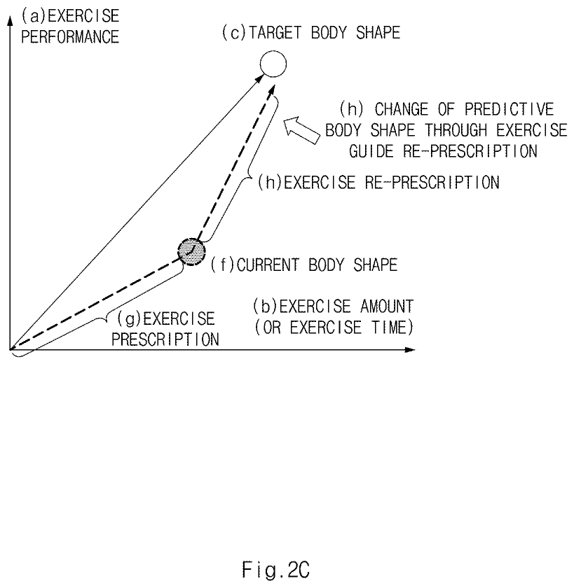

FIG. 1 is a block diagram illustrating an exercise prescription apparatus according to an embodiment of the present disclosure and FIGS. 2A to 2C are graphs of relationship between a target body shape and a predicted body shape associated with the present disclosure.

Referring to FIG. 1, an exercise prescription apparatus 100 includes a body shape measurement device 110, a user input device 120, a communication device 130, a storage 140, an output device 150, and a processor 160.

The body shape measurement device 110 of the exercise prescription apparatus 100 measures a user's body shape (body size) using, for example, a camera, a body scanner, and/or a vision sensor. The body shape measurement device 110 may extract a size of each body part (measurement), i.e., body shape data, such as shoulder width, waist width, pelvic width, and/or thigh width from an image acquired through a camera, a body scanner, and/or a vision sensor.

The user input device 120 of the exercise prescription apparatus 100 generates data based on a user's operation. The user input device 120 may be embodied as, for example, a keyboard, keypad, button, switch, touch pad, and/or touch screen. The user input device 120 may generate personal requirements, personal information, body shape information, and/or exercise information of the user based on a user input. The personal requirements are definitions of body characteristics that the user wants, such as, making a face look smaller or legs look longer. The personal information may include age, gender, height and/or exercise history. The body shape information includes body-specific measurement (e.g., height, arm length, leg length, upper body length, lower body length, and/or the like) and shape (e.g., appearance) information. The exercise information includes, for example, an exercise type (e.g., walking, running, biking and swimming, and the like), exercise intensity and/or exercise time.

The communication device 130 of the exercise prescription apparatus 100 may perform communication with an external device (e.g., a smart phone, a tablet, and/or a laptop computer). The communication device 130 may receive the personal requirements, personal information, body shape information, and/or exercise information of the user transmitted from the external device. The communication device 130 may use at least one of communication technologies such as wireless Internet (e.g., Wi-Fi™), wired Internet (e.g., Local Area Network (LAN) and Ethernet), local area communication (e.g., Bluetooth™, ZigBee®, and infrared communication), or mobile communication.

The storage 140 of the exercise prescription apparatus 100 may store software programmed to allow the processor 160 to perform a predetermined operation. The storage 140 may be implemented as at least one storage medium (recording medium), which is non-statutory, such as a flash memory, hard disk, Secure Digital (SD) card, random access memory (RAM), static random access memory (SRAM), and read only memory (ROM), programmable read only memory (PROM), electrically erasable and programmable ROM (EEPROM), erasable and programmable ROM (EPROM), registers, removable disks, and web storage.

The storage 140 may store the user's personal requirements, personal information, target body shape information and/or predicted body shape information (future body shape information) and may store the user's personal body shape history and/or exercise history. The personal body shape history (body shape history information) includes body shape data (body shape information) based on time measured by the body shape measurement device 110. The exercise history (exercise history information) includes exercise information (exercise record) performed by the user in chronological order, for example, date, exercise type, exercise time, distance, calorie consumption and/or average speed.

The output device 150 of the exercise prescription apparatus 100, which is for outputting information such as visual information, auditory information and/or tactile information, may include, for example, a display, a sound output module, and/or a tactile signal output module. The display may include at least one of a liquid crystal display (LCD), a thin film transistor-liquid crystal display (TFT-LCD), an organic light-emitting diode (OLED) display, a flexible display, a 3D display, a transparent display, a head-up display (HUD), or a touch screen. The audio output module may output audio data stored in the storage 140. The sound output module may include a receiver, a speaker, and/or a buzzer. The tactile signal output module may output a tactile signal (e.g., vibration).

The output device 150 may output a user's body shape information, i.e., a 2D or 3D avatar based on body measurement in accordance with instruction of the processor 160. The output device 150 may output at least one of a user's initial body shape avatar, current body shape avatar, or future body shape avatar (predicted body shape avatar). In other words, the output device 150 may simultaneously output the initial body shape avatar and the current body shape avatar, may simultaneously output the initial body shape avatar and the predicted body shape avatar, may simultaneously output the current body shape avatar and the predicted body shape avatar, or may simultaneously output the initial body shape avatar, the current body shape avatar, and the predicted body shape avatar. When outputting two or more avatars, the output device 150 may display two or more avatars overlapped. According to this embodiment, because two or more avatars are output at the same time, the user may intuitively compare body shapes through the output avatars. The output device 150 may output a user's body shape change in a form of a graph based on the personal body shape history, to allow the user to confirm the user's body shape change.

According to one exemplary embodiment of the present disclosure, the processor 160 of the exercise prescription apparatus 100 may control an overall operation of the exercise prescription apparatus 100. The processor 160 may be implemented as at least one of an application specific integrated circuit (ASIC), a digital signal processor (DSP), programmable logic devices (PLDs), field programmable gate arrays (FPGAs), a central processing unit (CPU), microcontrollers, or microprocessors.

According to one exemplary embodiment, the processor 160 may set or determine a target body shape based on one or more personal requirements of the user. When the personal requirements are input, the processor 160 may determine a body part to be changed, i.e., a part requiring exercise (a part requiring exercise) in consideration of the personal requirements. For example, when the user inputs a voluminous body shape as a personal requirement, the processor 160 determines a chest and hips as the exercise-requiring parts based on the inputted personal requirement. In addition, the processor 160 may determine at least one target body shape candidate (e.g., a slim body shape, a sports model type, or a bodybuilder type) based on a range in which a body shape change is possible for a portion requiring exercise. The range of body shape change may be limited by personal information such as age, gender, exercise history, and/or height of the user. The processor 160 may output at least one target body shape candidate determined to be intuitively understood by the user as an avatar of 2D or 3D graphics. The processor 160 may select any one of the at least one target body shape candidate based on the user input as a final target body shape. The processor 160 may adjust a size (parameters such as length, width, and/or circumference) of each body part of the target body shape that is finally selected based on the user input. In addition, when the user's body shape information (initial body shape information) is input, the processor 160 may reflect basic skeleton information (including height, upper body length, lower body length, and/or arm length) extracted from the user's body shape information when setting the target body shape. Therefore, when the target body shape is determined, an ideal body shape may not be set as the target body shape, but a realistic body shape may be set as the target body shape.

According to one exemplary embodiment, the processor 160 may determine an exercise prescription based on the user's initial body shape information and/or target body shape. The exercise prescription may include exercise type, exercise intensity, exercise time, exercise frequency, and exercise duration. Subsequently, the processor 160 may record a user's exercise activity in accordance with an exercise prescription using a position measurement sensor (not illustrated) and a heart rate measurement sensor (not illustrated). In addition, the processor 160 measures and records the change in the user's body shape over time through the body shape measurement device 110. The processor 160 measures the user's current body shape through the body shape measurement device 110 and generates and outputs the current body shape avatar using the measurement data. The processor 160 generates exercise history information through a record of exercise activity and generates body shape history information through measurement of the user's body shape change.

According to one exemplary embodiment, the processor 160 may predict or estimate the user's future body shape (predicted body shape) based on the body shape history information and/or exercise history information. When predicting the future body shape, the processor 160 may use a body shape history of another person as well as the personal body shape history to group body shapes based on body characteristics (e.g., physical characteristics), and analyze the grouped body characteristics based on data reflecting the correlation between exercise and body shape among grouped body characteristics to confirm the body shape change characteristics for each the body characteristics and for each group. The processor 160 may predict the user's future body shape utilizing the confirmed body shape change characteristics. The processor 160 may shape and output the predicted future body shape as an avatar. Therefore, the user may intuitively confirm the future body shape.

According to one exemplary embodiment, the processor 160 may compare the predicted future body shape with the set target body shape to analyze a difference in body shape and determines whether exercise re-prescription is necessary based on an analysis result. As illustrated in FIG. 2A, the processor 160 determines that exercise re-prescription is unnecessary when the predicted body shape matches the target body shape. As illustrated in FIG. 2B, the processor 160 determines that exercise re-prescription is necessary when there is a mismatch between the predicted body shape and the target body shape. Accordingly, the processor 160 re-prescribes an exercise guide based on the user's current body shape information and target body shape. The processor 160 corrects the previous exercise prescription to minimize the difference between the predicted body shape and the target body shape as illustrated in FIG. 2C.

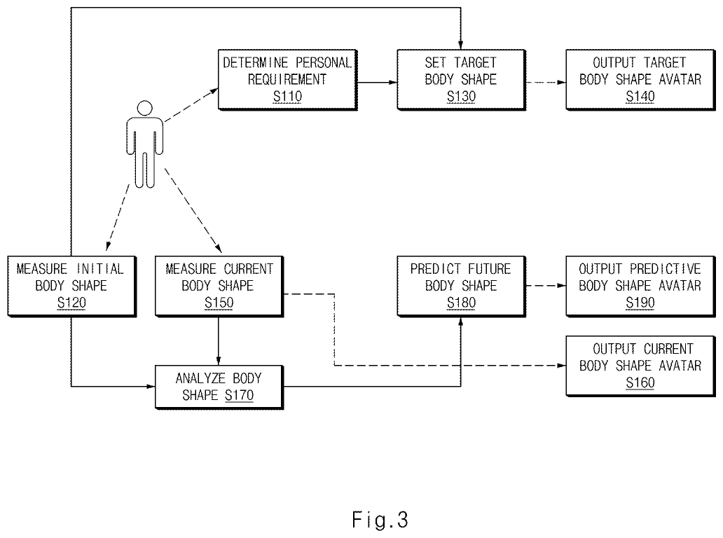

FIG. 3 is a flowchart illustrating a method of predicting a user body shape according to an embodiment of the present disclosure.

According to one exemplary embodiment, the processor 160 of the exercise prescription apparatus 100 may determine one ore more personal requirements when the one or more personal requirements are input from the user in S110. In other words, the processor 160 may determine a body part in need of exercise, i.e., a part in need of exercise based on the personal requirements. Here, three personal requirements are definitions of body characteristics desired by the user.

According to one exemplary embodiment, the body shape measurement device 110 may measure an initial body shape of the user in S120. The processor 160 may extract basic skeleton information of the user from the measured initial body shape of the user.

According to one exemplary embodiment, the processor 160 may set the target body shape based on the personal requirements in S130. The processor 160 may reflect the user's body shape changeable range and/or basic skeleton information when setting the target body shape. Here, the body shape changeable range may be limited by personal information such as age, gender, exercise history, and/or height.

According to one exemplary embodiment, the processor 160 may shape and output the set target body shape as an avatar in S140. The user may confirm the target body shape set intuitively through the avatar. After confirming the target body shape avatar, the processor 160 may adjust the size of each body part of the target body shape based on the user input to correct the target body shape.

When the target body shape is set, the processor 160 may determine the exercise prescription based on the initial body shape information and/or target body shape information. The exercise prescription may include an exercise type, exercise intensity, exercise time, exercise frequency, and exercise period. The user may perform the exercise based on the exercise prescription and may record information about the exercise activity, such as exercise type, exercise intensity, exercise time, and/or exercise amount.

According to one exemplary embodiment, the processor 160 may measure the user's current body shape using the body shape measurement device 110 in S150. The processor 160 may measure and record the change in body shape of the user who performs the exercise based on the exercise prescription. In other words, the processor 160 may store the body shape information, i.e., the body shape history information, over time in the storage 140.

According to one exemplary embodiment, the processor 160 may generate and output an avatar based on the measured current body shape information in S160. The user may intuitively confirm the current body shape through the visually displayed avatar.

According to one exemplary embodiment, the processor 160 may analyze the change in the user's body shape using the initial body shape information and the current body shape information in S170. The processor 160 may analyze a user's body shape change trend based on the body shape history information. In addition, the processor 160 may group at least one other person having a body characteristics similar to the user's body characteristics with a predetermined ratio or more using the user's personal body shape history as well as the other body shape history. The processor 160 analyzes the trend of changes in the body shape of the user based on the data reflecting the correlation between exercise and body shape among the body characteristics of the grouped user and others. The processor 160 may analyze the correlation between exercise and body shape based on the user's body shape history information and exercise history information.

According to one exemplary embodiment, the processor 160 may predict or estimate the future body shape based on the analysis result in S180. The processor 160 may predict the future body shape, i.e., the change of the user's body shape, based on the analyzed body shape change trend.

When the future body shape (predicted body shape) is predicted, the processor 160 generates and outputs an avatar based on the predicted body shape information in S190. The processor 160 may generate the avatar based on the size information of each body part of the predicted future body shape.

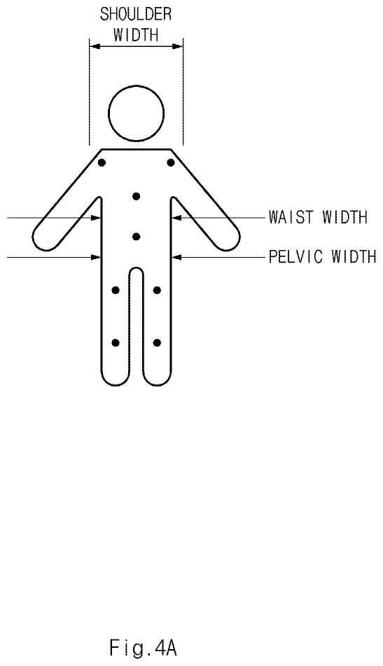

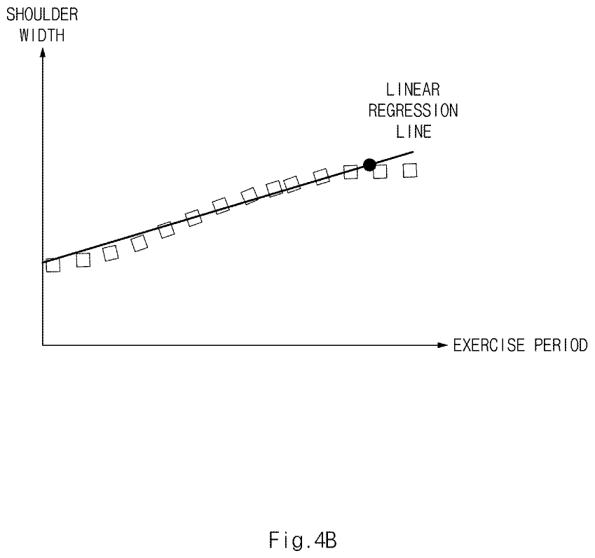

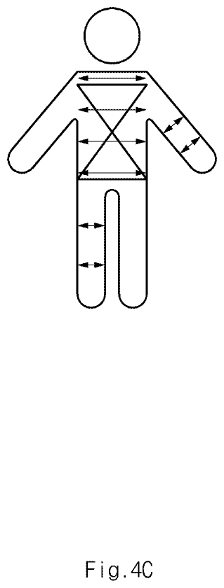

FIGS. 4A to 4C are diagrams for explaining future body shape prediction depending on exercise according to an embodiment of the present disclosure.

A body shape may be composed of a number of body parts (body portions) and may confirm the trend of body shape change of the entire body by confirming the trend of the size change of each body part over time.

Referring to FIG. 4A, the processor 160 may determine a shoulder width and a pelvic width based on a point where a torso and limbs are connected and may measure a waist width at a certain percentage point of shoulder and pelvic joints (e.g., ⅓ point). A size change pattern of each body part varies depending on the user's initial body shape, exercise method, exercise type, and muscle characteristics. That is, for each user, a size increase/decrease pattern of each body part, i.e., a slope of a size increase/decrease trend line and an initial size of each body part are different. The trend line may be defined as a linear regression line as illustrated in FIG. 4B or a polynomial equation. Also, as a trend line, a trend function having a minimum error value may be selected depending on a data pattern.

The processor 160 may estimate the future size (dimension) of each body part through the size trend line for each body part of the user. The processor 160 may predict the size of the entire body shape by combining the predicted future size of each body part.

The processor 160 may determine an appearance type by a combination of the future appearance size of each body part. The appearance type (body shape) is divided into an inverted triangle, a square and an hourglass based on the shoulder width, waist width, and pelvic width.

For example, as illustrated in FIG. 4C, when the shoulder width is greater than a predetermined ratio compared to the waist width and the pelvic width is greater than a predetermined ratio compared to the waist width, the processor 160 determines the body shape as the hourglass type.

Alternatively, the processor 160 may determine the body shape as the inverted triangle when the waist width and the pelvic width are significantly narrow compared to the shoulder width and determine the body shape as the square when the shoulder width, waist width, and pelvic width are similar each other.

Further, when the size of each body part changes over time and reaches an increase/decrease maximum limit value determined by a user's body shape statistics, the processor 160 determines the corresponding maximum limit value as a size of a matched body part. When the size of each body part converges to a specific value, the processor 160 may determine a convergence value as a size of a matched body part. The processor 160 may determine the size of each body part as a predicted dimension at a predetermined time. The increase/decrease maximum limit value of each body part, the predicted dimension and convergence value at a predetermined time point are factors that determine the shape of the overall body shape.

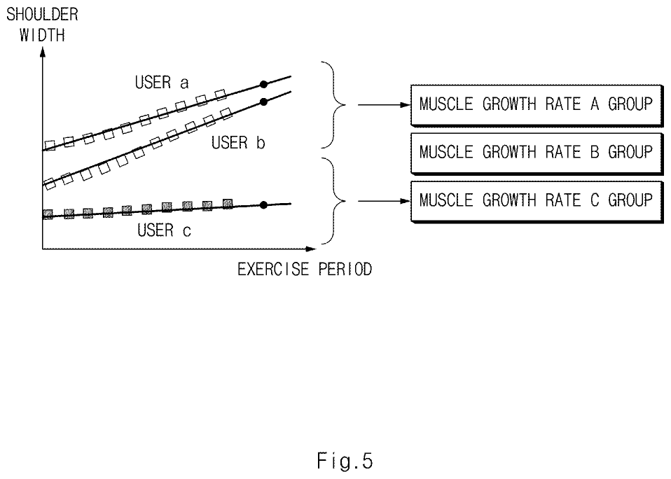

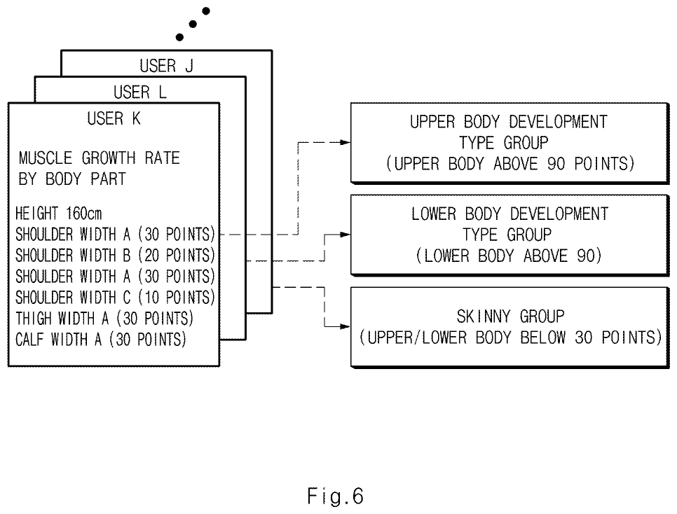

FIGS. 5 and 6 are diagrams for explaining grouping based on body characteristics according to an embodiment of the present disclosure.

Deviation may occur in the size change and body shape change of each body part among users although the exercise is performed based on the same exercise guide because a size increase/decrease rate and body shape change of each body part based on a user's constitution and an actual exercise manner are different. As illustrated in FIG. 5, although the same exercise is performed for the same period, an increase rate in shoulder width is different for each user. The processor 160 may classify a user into an A group, a B group, and a C group depending on a muscle growth rate, i.e., the shoulder width increase rate, of the user and at least one or more others. In addition, the processor 160 may score each body part based on the muscle growth rate of each body part for each user and determine body characteristics for each user and groups users. As illustrated in FIG. 6, the processor 160 may group the user's body shape characteristics into an upper body development type, a lower body development type, or a skinny type based on scores for each body part. When prescribing an exercise guide, the processor 160 may customize and correct the exercise guide depending on the grouped body characteristics. The muscle growth rate of each part of the body of the grouped users is similar, but the predicted future body shape may vary significantly depending on the user's exercise type, exercise cycle, and exercise time.

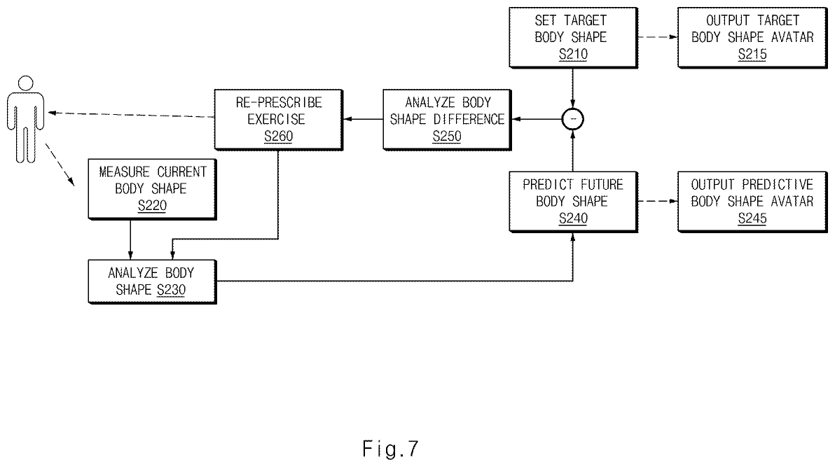

FIG. 7 is a flowchart illustrating a method of prescribing exercise according to an embodiment of the present disclosure.

When the target body shape is set by the user in S210, the processor 160 may generate and output the target body shape avatar in S215. The processor 160 may set the target body shape based on the user's personal requirements. The processor 160 may set a target body shape based on the user's personal requirements and basic skeleton information.

The processor 160 may continuously measure the body shape of the user in S220. The processor 160 stores the measured body data (body information) in a time order in the storage 140. The processor 160 may measure the size of each user's body part at a predetermined cycle. The processor 160 may grasp the user's body shape by combining the measured sizes of each body part.

The processor 160 may analyze the user's body shape by continuously analyzing the measured body shape information in S230. The processor 160 may confirm the trend of the user's body shape change based on the user's body shape history information.

The processor 160 may predict the future body shape based on an analysis result in S240. The processor 160 may estimate the user's future body shape based on the user's body shape change trend.

The processor 160 may generate and output the predicted future body shape, i.e., the predicted body shape as an avatar in S245. The user may confirm the future body shape through the predicted body shape avatar displayed.

The processor 160 may compare the predicted future body shape with the target body shape to analyze the difference in body shape in S250. The body shape difference information, which is a difference between the predicted size and target size for each body part, may include a length difference for each body part, a circumference difference, and/or a posture difference (skeleton difference).

The processor 160 may re-prescribe the exercise guide in consideration of the analyzed body shape difference in S260. The processor 160 may prescribe an exercise guide to compensate for the difference between the predicted body shape and the target body shape. That is, the processor 160 corrects the previous exercise prescription in consideration of the difference between the predicted body shape and the target body shape. The processor 160 continuously stores the difference information between the predicted body shape and the target body shape and the exercise prescription information in the storage 140 to use the difference information and the exercise prescription information with the current body shape information and the target body shape information as main parameters when predicting the future body shape.

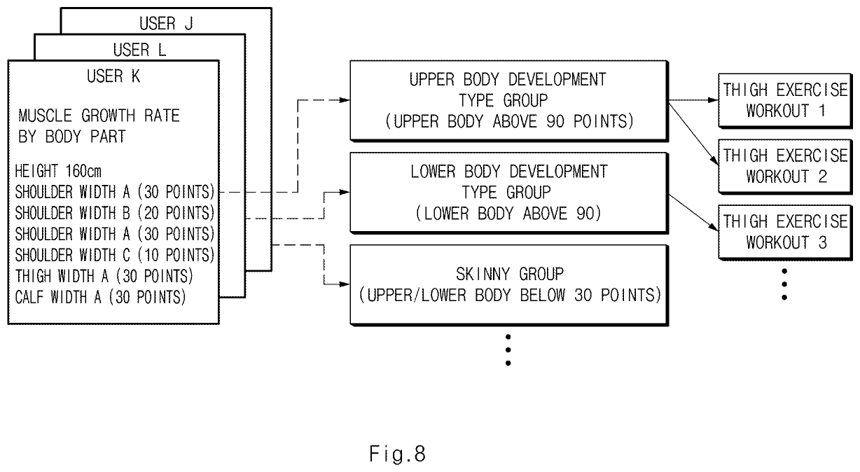

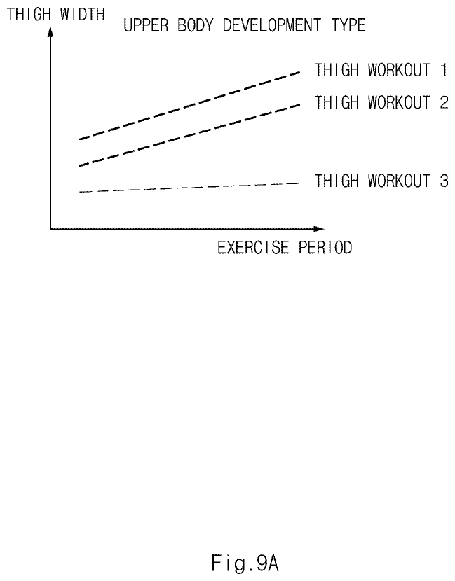

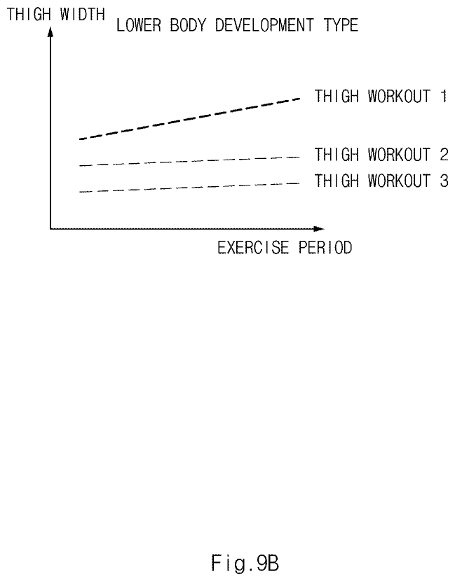

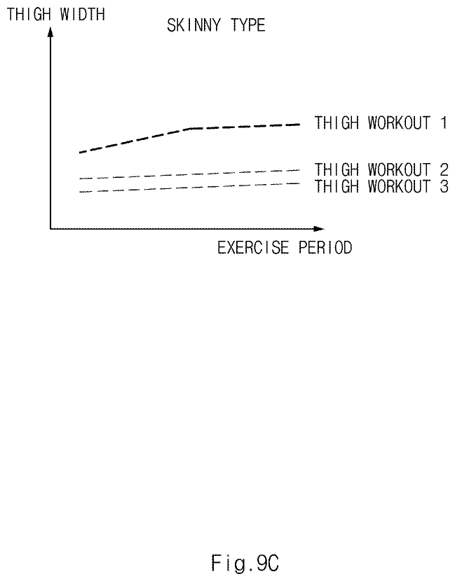

FIG. 8 is a view for explaining an exercise prescription for each body shape characteristics according to an embodiment of the present disclosure, and FIGS. 9A to 9C are graphs illustrating a correlation between body shape characteristics and an exercise type according to an embodiment of the present disclosure.

The processor 160 of the exercise prescription apparatus 100 may prescribe a customized exercise guide reflecting the grouped body characteristics of users. For example, when the user belonging to the upper body development type group sets the target body shape having uniform muscle distribution of the entire body, the processor 160 allocates more types and times of exercises related to the lower body than exercises related to the upper body. In addition, although the processor 160 prescribes same thigh exercise, i.e., lower body strengthening exercise, an exercise having a better muscle mass increase rate depending on the body shape characteristics is continuously prescribed. For example, as illustrated in FIG. 8, workout 1 and workout 2 of the thigh exercise type are prescribed to a user belonging to the upper body development type group, and workout 3 of the thigh exercise type is prescribed to a user belonging to the lower body development type group. Here, the processor 160 prescribes an exercise manner having a better exercise effect over time in consideration of an increase rate of thigh width (muscle amount) depending on the exercise type based on the body shape characteristics illustrated in FIGS. 9A to 9C. In other words, the processor 160 may record and statistically process effects on the exercise depending on the body characteristics to prescribe the exercise having the better exercise effect compared to the same time.

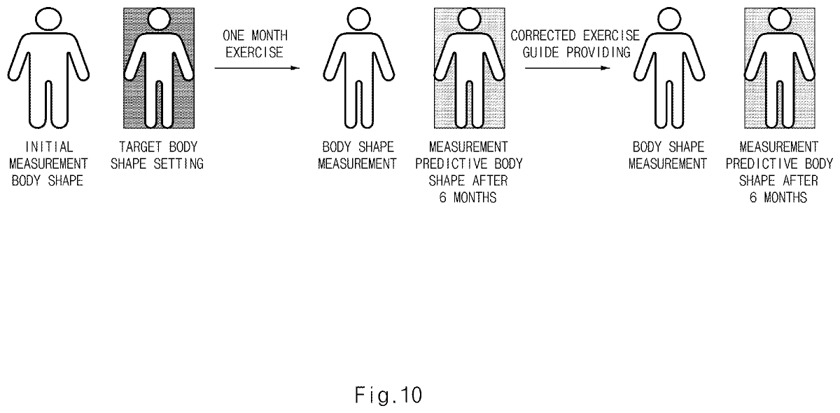

FIG. 10 is a view for explaining a method of prescribing exercise according to another embodiment of the present disclosure.

The exercise prescription apparatus 100 measures the user's current body dimension, i.e., an initial body shape, through a vision-based body appearance measuring device (not illustrated). In addition, the exercise prescription apparatus 100 sets the target body shape based on the basic skeleton information of the user. The exercise prescription apparatus 100 prescribes the exercise guide based on the initial body shape information and target body shape information. The user performs the exercise for a certain period (e.g., 1 month) based on the exercise prescription.

After the exercise is performed for a certain period of time, the exercise prescription apparatus 100 measures the user's current body shape using the body appearance measuring device (not illustrated). Then, the exercise prescription apparatus 100 predicts the future body shape after a predetermined period (e.g., 6 months) based on the initial body shape and the current body shape. The exercise prescription apparatus 100 compares the predicted future body shape and the target body shape to analyze the difference between the two body shapes. The exercise prescription apparatus 100 corrects an existing exercise guide in consideration of the difference between the future body shape and the target body shape and provides the corrected exercise guide to the user. Thereafter, the exercise prescription apparatus 100 compares the user's measured body shape and the next future body shape after the user performs exercise for a predetermined period based on the corrected exercise guide, and re-correct the exercise guide when there is a difference between the two body shapes.

As this embodiment, the future body shape may be predicted, and the existing exercise prescription may be corrected and provided in consideration of the difference between the predicted body shape and the target body shape, and may minimize the difference between the changed body shape of the user who performed the exercise based on the exercise prescription and the target body shape.

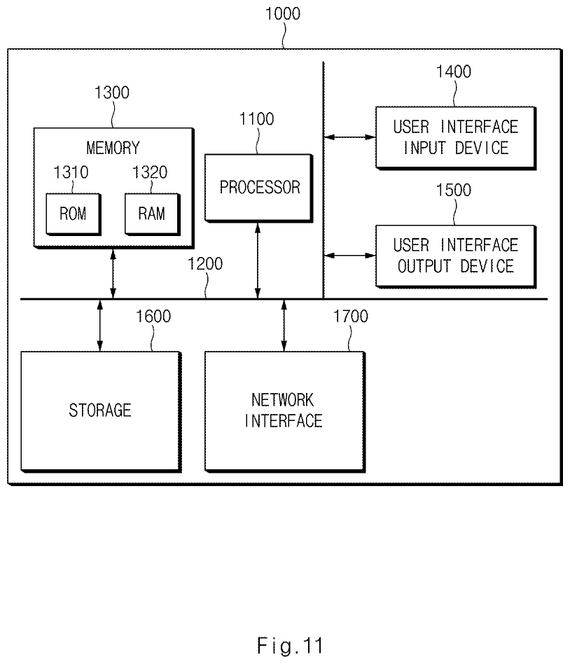

FIG. 11 is a block diagram illustrating a computing system for executing a method of prescribing exercise according to an embodiment of the present disclosure.

Referring to FIG. 11, a computing system 1000 may include at least one processor 1100, a memory 1300, a user interface input device 1400, a user interface output device 1500, storage 1600, and a network interface 1700, which are connected with each other via a bus 1200.

The processor 1100 may be a central processing unit (CPU) or a semiconductor device that processes instructions stored in the memory 1300 and/or the storage 1600. The memory 1300 and the storage 1600 may include various types of volatile or non-volatile storage media. For example, the memory 1300 may include a ROM (Read Only Memory) 1310 and a RAM (Random Access Memory) 1320.

Thus, the operations of the method or the algorithm described in connection with the embodiments disclosed herein may be embodied directly in hardware or a software module executed by the processor 1100, or in a combination thereof. The software module may reside on a storage medium (that is, the memory 1300 and/or the storage 1600) such as a RAM, a flash memory, a ROM, an EPROM, an EEPROM, a register, a hard disk, a removable disk, a CD-ROM. The exemplary storage medium may be coupled to the processor 1100, and the processor 1100 may read information out of the storage medium and may record information in the storage medium. Alternatively, the storage medium may be integrated with the processor 1100. The processor 1100 and the storage medium may reside in an application specific integrated circuit (ASIC). The ASIC may reside within a user terminal. In another case, the processor 1100 and the storage medium may reside in the user terminal as separate components.

According to exemplary embodiments of the present disclosure, the body shape change of the user may be predicted based on the user's body shape change information and exercise history information and the exercise guide may be prescribed in consideration of the difference between the predicted body shape and the target body shape, to provide the user with the necessary exercise correction to reach the target body shape in a timely manner.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims. Therefore, the exemplary embodiments of the present disclosure are provided to explain the spirit and scope of the present disclosure, but not to limit them, so that the spirit and scope of the present disclosure is not limited by the embodiments. The scope of the present disclosure should be construed on the basis of the accompanying claims, and all the technical ideas within the scope equivalent to the claims should be included in the scope of the present disclosure.

What is claimed is:

1. An exercise prescription apparatus comprising:
a body shape measurement device configured to measure a body shape of a user changing with a progress of an exercise based on a previously prescribed exercise guide; and
a processor configured to predict a future body shape of the user based on body shape history information obtained through the measured body shape of the user and to re-prescribe an exercise guide based on a body shape difference between the predicted future body shape and a target body shape.

2. The exercise prescription apparatus of claim 1, wherein the processor determines one or more personal requirements of the user to set the target body shape.

3. The exercise prescription apparatus of claim 2, wherein the processor extracts basic skeleton information through a measurement of an initial body shape of the user by the body shape measurement device and reflects the extracted basic skeleton information in the target body shape.

4. The exercise prescription apparatus of claim 1, wherein the processor confirms a body shape change trend of the user based on the body shape history information and predicts the future body shape based on the confirmed body shape change trend.

5. The exercise prescription apparatus of claim 1, wherein the processor predicts the future body shape based on an exercise type, an exercise intensity, an exercise time, an exercise frequency, and an exercise period of the user.

6. The exercise prescription apparatus of claim 1, wherein the processor groups at least one other person having a similar body characteristics to a body characteristics of the user with a predetermined ratio or more, and predicts the future body shape based on the body shape history information of the user and the at least one other person which are grouped.

7. The exercise prescription apparatus of claim 6, wherein the body characteristics include a dimensional increase/decrease rate for each body part.

8. The exercise prescription apparatus of claim 1, wherein the processor predicts a body appearance type of the user using an increase/decrease maximum limit value of a size of each body part, a convergence value, and a prediction value at a predetermined time.

9. The exercise prescription apparatus of claim 1, wherein the processor shapes and outputs at least one of the measured body shape, the future body shape, or the target body shape as an avatar.

10. A method of prescribing an exercise comprising:
measuring a body shape of a user which changes with a progress of an exercise based on a previously prescribed exercise guide;
predicting a future body shape of the user based on body shape history information obtained through the measured body shape of the user; and
re-prescribing an exercise guide based on a difference in body shape between the predicted future body shape and a target body shape.

11. The method of prescribing an exercise of claim 10, wherein the measuring a body shape of a user includes determining one or more personal requirements of the user to set the target body shape.

12. The method of prescribing an exercise of claim 11, wherein the measuring a body shape of a user further includes measuring an initial body shape of the user to extract basic skeleton information and reflecting the extracted basic skeleton information in the target body shape.

13. The method of prescribing an exercise of claim 10, wherein the predicting a future body shape of the user includes confirming a body shape change trend of the user based on the body shape history information and reflecting the confirmed body shape change trend to predict the future body shape.

14. The method of prescribing an exercise of claim 10, wherein the predicting a future body shape of the user includes grouping at least one other person having a similar body characteristics to a body characteristics of the user with a predetermined ratio or more, and predicting the future body shape based on the body shape history information of the user and the at least one other person which are grouped.

15. The method of prescribing an exercise of claim 10, wherein the predicting a future body shape of the user includes predicting a body appearance type of the user using an increase/decrease maximum limit value of a size of each body part, a convergence value, and a prediction value at a predetermined time.

16. The method of prescribing an exercise of claim 10, wherein the predicting a future body shape of the user includes predicting the future body shape of the user based on an exercise type, an exercise intensity, an exercise time, exercise frequency, and an exercise period of the user.

17. The method of prescribing an exercise of claim 10, further comprising
shaping and outputting at least one of the measured body shape, the future body shape, or the target body shape of the user as an avatar.
